(19) United States
(12) Patent Application Publication
(10) Pub. No.: US 2010/0324331 A1
(43) Pub. Date: Dec. 23, 2010

(54) MIXED OXIDE CATALYSTS MADE OF HOLLOW SHAPES

(76) Inventors: Achim Fischer, Aschaffenburg (DE); Werner Burkhardt, Brachttal (DE); Stefan Röder, Sinntal (DE); Klaus Huthmacher, Gelnhausen (DE)

Correspondence Address: SMITH, GAMBRELL & RUSSELL, 1130 CONNECTICUT AVENUE, N.W., SUITE 1130, WASHINGTON, DC, 20036, US

(21) Appl. No.: 12/528,407

(22) PCT Filed: Jan. 28, 2008
(86) PCT No.: PCT/EP2008/050985

§ 371 Date: Aug. 24, 2009

(30) Foreign Application Priority Data
Mar. 1, 2007 (DE) 10 2007 009 981.0

(51) Int. Cl. C07C 51/265; B01J 27/192; B01J 23/28; C07C 51/25; C07C 45/32

(52) U.S. Cl. 562/415; 502/212; 502/311; 562/412; 562/547; 568/431

(57) ABSTRACT

The invention relates to mixed oxide catalysts made of hollow shapes for the catalytic gas phase oxidation of olefins, and to a method for producing the catalysts by applying them as a layer to a carrier made of organic material and removing said organic material. The reaction into aldehydes and carboxylic acids occurs by air or oxygen in the presence of inert gases in different quantity ratios, at elevated temperatures and pressure in the presence of said catalysts.

The invention relates to mixed oxide catalysts, consisting of hollow shapes, for the catalytic gas phase oxidation of olefins, to processes for preparing the catalysts and to the reaction to give aldehydes and carboxylic acids with air or oxygen in the presence of inert gases in different quantitative ratios, at elevated temperatures and pressures.

In particular, the catalyst can be used to implement the strongly exothermic reaction of propene to acrolein and acrylic acid or isobutene to methacrolein and methacrylic acid. The strongly exothermic reaction of the olefin over heterogeneous catalysts with an oxygen-comprising gas leads not only to the desired acrolein and acrylic acid product but also to a series of by-products: for example to the formation of CO₂, CO, acetaldehyde or acetic acid.

It is known that the type of chemical composition of the mixed oxide (phase formation and formation of reaction sites) and also the type of physical structure (for example porosity, surface size, shape of the catalyst) and the type of heat removal can greatly influence the ability to form products (selectivity) and the productivity (space-time yield). In the case of the olefin oxidation, the catalysts used are generally mixed oxides which, in their chemical and physical makeup, have a complex structure. A multitude of publications describe mixed oxides which are capable of being used as catalysts for the preparation of acrolein and acrylic acid from propene. These catalysts consist generally of molybdenum, vanadium and/or tungsten. Generally added to these base components is at least one of the elements bismuth, antimony, vanadium, tellurium, tin, iron, cobalt, nickel and/or copper.

The number of publications regarding heterogeneously catalysed gas phase oxidation of olefins to acrolein and acrylic acid is numerous since the first development GB 821999 (1958) to Standard Oil Inc. In spite of the long development time, it is still a demanding problem to improve the performance of the catalyst, such as product yield, activity and lifetime. For this purpose, the literature claims various techniques for preparation, and formulations of the catalyst. By way of example, the most recent developments are presented here:

### STATE OF THE ART

WO 2005/063673 relates to the dilution of the catalyst by an inert material in order to reduce the heat formation in the reaction zone and hence to increase the product yield. Avoidance of an excessively high temperature reduces the total oxidation of the products. In spite of the temperature modulation of the reaction by inerts, only accumulated yield of acrolein and acrylic acid of no more than 91.22% is achieved by the process described.

WO2005035115 describes the preparation of a catalyst of the metal oxide composition and sublimeable materials. The metal oxide acts as a catalyst; the sublimeable material acts as an additive for pore generation. The resulting catalyst is very active, has a large surface area and is capable of forming acrolein and acrylic acid with high selectivity.

DE 10344149 discloses an annular unsupported catalyst based on a Mo₁₂BiₐFe_{b}X1_{c}X2_{d}X3ₑX4_{f}Oₙ (1) with a length of 2-11 mm, an external diameter of 2-11 mm and a wall thickness of 0.75-1.75 mm for partial oxidation of propene to acrolein or methacrolein. Catalyst (I) has the advantage of an improved activity and selectivity.

DE 199 33450 describes metal catalysts which are composed of nickel, comprise hollow shapes or spheres composed of a metal alloy, and are used for hydrogenation, dehydrogenation, isomerization, reductive alkylation and reductive amination. The catalyst thus prepared has an improved stability and lifetime.

Such hollow spheres can be prepared according to Andersen, Schneider and Stephanie (cf. “Neue Hochporöse Metallische Werkstoffe” [Novel highly porous metallic materials], Ingenieur-Werkstoffe, 4, 1998, p. 36-38). In this process, a mixture of the desired alloy, of an organic binder and optionally of an inorganic binder is sprayed uniformly through a fluidized bed composed of polystyrene spheres, where it coats the spheres. The coated spheres are then calcined at selected temperatures within the range of 450 to 1000° C. in order to burn out the polystyrene, followed by a higher calcination temperature in order to sinter the metal together and stabilize the hollow shape. After the calcination, the catalyst is activated by a sodium hydroxide solution in order to prepare the activated base metal catalyst. An additional advantage of this catalyst system is that the thickness of the walls of the hollow shapes can be controlled easily through the coating conditions, and the porosity of the walls through the particle size and composition of the original powder mixture.

### PROBLEM

It is generally accepted that the type of chemical composition of the mixed oxide (phase formation and formation of reaction sites) and the type of physical structure (for example porosity, surface size, shape of the catalyst) and the type of heat removal can greatly influence the ability to form products (selectivity) and the productivity (space-time yield). The present invention has for its object to provide a catalyst with an elevated catalytic activity compared to the prior art.

The invention is based on the further object of providing an improved process for preparing aldehydes and acids, in which acrolein and acrylic acid are prepared from propene by oxidation with air or oxygen in the presence of inert gases, including steam or offgases from the reaction, at elevated temperatures and in the presence of a heterogeneous mixed oxide catalyst. A mixed oxide catalyst shall be provided, with which not only propene conversions greater than 95% but, also a high product selectivity of greater than or equal to 88% are achieved, such that the economic viability of the process is improved.

### DESCRIPTION OF THE INVENTION

The invention provides catalysts, consisting of hollow shapes, for oxidizing olefins, for example mixed oxide catalysts of the general formula

(Mo₁₂BiₐC_{b}(Co+Ni)_{c}DₐEₑF_{f}G_{g}Hₕ)Oₓ (I)

in which
C: iron,
D: at least one of the elements selected from W, P,
E: at least one of the elements selected from Li, K, Na, Rb, Cs, Mg, Ca, Ba, Sr,
F: at least one of the elements selected from Ce, Mn, Cr,
V,
G: at least one of the elements selected from Nb, Se, Te, Sm, Gd, La, Y, Pd, Pt, Ru, Ag, Au,
H: at least one of the elements selected from Si, Al, Ti, Zr,
   and
   a=0.5-5.0
   b=0.5-5.0
   c=2-15
   d=0.01-5.0
   e=0.001-2
   f=0.001-5
   g=0-1.5
   h=0-800,
   and
   x=number which is determined by the valency and frequency of the elements other than oxygen.

The use of the inventive catalysts leads to a significantly improved catalyst activity which is manifested in that lower salt bath temperatures can be established for high conversions.

As a result of the novel process for preparing the catalysts, for example of the general formula I, it is possible to obtain a particularly suitable catalytically active solid, for example for converting propene to acrolein and acrylic acid. The reaction is particularly advantageously performed in reactors which allow the catalyst to be used as a fixed bed. However, it is likewise possible to use the catalyst as a fluidized bed catalyst. It should be pointed out here that the inventive catalysts can also be utilized for the conversion of isobutene to methacrolein and methacrylic acid.

Catalysts of the composition described can be prepared by obtaining a finely divided powder by the production steps of: dissolving the metal salts, precipitating the active components, drying and Calcination, and shaping the calcined powder. This can be done in the commonly known manner by tableting, extrusion or by, coating of a support. The support shape is not limiting. For example, the support may be a pyramid, a cylinder or a sphere.

A novel process has now been found in order to give the mixed oxide catalyst a hollow shape. In this case, the support is a matrix which imparts a shape to the active composition and is removed after or during the solidification of the active composition so as to form a hollow body. The removal is effected by controlled leaching-out by means of a solvent or preferably thermally, for example by means of thermal radiation. The coated support should preferably be treated in the temperature range of 450 to 600° C. in the presence of oxygen, especially of air, such that the catalytically active composition for use in industrial reactors solidifies and the support decomposes without residue. These supports used are organic materials, for example polystyrene-based polymers such as ASA (acrylonitrile/styrene/acrylic ester), polystyrene (PS, PS-I), SAN (styrene/acrylonitile). However, there is no restriction to these polymers. These materials are generally significantly cheaper than the ceramic supports, such that the preparation costs of the catalyst are reduced.

The size of the support matrix is not limiting. Typically, bodies of 0.1 to 20 mm, especially to 5 mm, are used. It is also conceivable to use supports in the range of 10⁻⁶ to 0.1 mm or greater than 2 mm.

The catalyst thus prepared has an excellent activity at high selectivity and lifetime and leads to a very good product yield.

The catalysts to be used for gas phase oxidation in the process described are obtained by combining the dissolved compounds of the catalytically active elements from the formula I with the desired concentrations. The components are used ideally in the form of the compounds selected from the group of ammonium or amine compounds, oxalates, carbonates, phosphates, acetates, carbonyls and/or nitrates, individually or together. Particular preference is given to carbonates, nitrates and phosphates or mixtures thereof. It is likewise possible to use acids of the salts, for example nitric acid, phosphoric acid or carbonic acid.

The first stage of the catalyst preparation forms, as already mentioned, a precipitate. Depending on the type of metal salts which are used in the precipitation stage, it may be necessary to add the components to the precipitation mixture in the form of solution mixtures. Ideally, ammonia or ammonium salts are used here, for example ammonium carbonate, ammonium heptamolybdate or metal nitrates, for example iron nitrate, cobalt nitrate; it is likewise possible to use the corresponding acids, for example nitric acid, in the amounts needed to establish the ionic ratio. The pH during the precipitation is <8, especially <7.

The preparation of coprecipitates can be performed in one precipitation stage. It is particularly preferred to perform the precipitation in several stages through stepwise addition of the individual components or through mixtures thereof. The number of precipitation stages is not limited in principle. However, preference is given to one to three precipitation stages.

The resulting suspension can be processed further directly, or it is allowed to mature for >0 to 24 hours, preferably >0 to 12 hours, more preferably >0 to 6 hours. It is obvious that the precipitated suspension, before the further processing, is homogenized, for example by stirring.

After the maturing, the liquid can be removed from the suspension by evaporation, centrifugation or filtration. It is likewise possible to evaporate the liquid and simultaneously to dry the solid, which can be effected, for example, by spray-drying. The liquid should be evaporated at a temperature of 80 to 130° C. The solid can be dried with air, oxygenous inert gases or inert gases, for example nitrogen. When the drying is performed in an oven, the temperature should be between 100 and 200° C. In a spray-dryer, the starting temperature of the drying medium should be from 200 to 500° C., and a temperature on deposition of the dried powder of from 80 to 200° C. Should be provided.

The resulting particles should be preferably have a particle size distribution of 15 to 160 μm with a mean particle diameter between 15 and 80 μm.

The dried powder may in principle subsequently be calcined in a wide variety of different oven types, for example in a forced-air oven, rotary oven, tray oven, shaft oven or belt oven. The control quality and the quality of temperature detection of the oven should be at a maximum. The residence time of the powder in the oven should, according to the oven type, be between 0.25 and 13 h.

It is likewise possible to perform the calcination and the thermal decomposition of the salts, for example nitrates or carbonates, which occurs at the same time in one or more stages. It is possible to employ temperatures of 200 to 600° C., especially 300 to 600° C. The thermal decomposition can be performed with addition of inert gas, composed of mixtures of oxygen with an inert gas.

Useable inert gases are, for example, nitrogen, helium, steam or mixtures of these gases.

The powder thus obtained may be used directly as a catalyst. The mean particle size distribution of the powder should range from 0.01 to 50 μm.

In order to convert the mixed oxide powder to the inventive form, it is applied to a support which, after the solidification of the catalytically active composition, is removed so as to form a hollow body. The removal is performed by controlled leaching-out by means of a solvent or preferably thermally, for example by thermal radiation. The precursor of the inventive catalyst, which consists of support and catalytically active layer, is preferably treated in the temperature range of 490 to 600° C., especially 490 to 580° C., such that the catalytically active composition for use in industrial reactors solidifies and the support can simultaneously or subsequently be removed without residue. The supports used are organic materials, for example polystyrene-based polymers such as ASA (acrylonitrile/styrene/acrylic ester), polystyrene (PS, PS-I), SAN (styrene/acrylonitrile), but there is no restriction to these polymers; it is also possible, for example, to use celluloses or sugars.

The geometric shape of the support is not limiting in this context. Instead, it is guided by the requirements of the reactor and of the reaction regime (for example tube diameter, length of the catalyst bed). For example, the support may be a pyramid, a cylinder, a saddle, a sphere or a polygon. Likewise not limiting is the size of the support. Typically, supports of 0.1 to 5 mm are used. However, it is also conceivable to use supports in the range of 10⁻⁶ to 0.1 mm or greater than 2 mm. The thickness of the mixed oxide layer is, according to the support size, generally between 10⁻⁶ and 1.5 mm; particular preference is given to a coating thickness of 0.1 to 1.5 mm. The coating of the support to prepare the catalyst precursor is performed by spraying an aqueous suspension which comprises the catalyst powder and binder. The catalyst powder is preferably used in a form calcined at 470° C. to 600° C. For the later formation of pores, one of the known pore formers may also be added to the suspension.

The binders used may be various oils, celluloses, polyvinyl alcohols, saccharides, acrylates and alkyl derivatives, mixtures or condensates thereof. Preference is given to acrylates, polyvinyl alcohols, and celluloses or sugars. Particular preference is given to derivatives and condensates of acrylates and/or celluloses and/or sugars, and mixtures thereof.

After drying of the coated support at temperatures of preferably up to 110° C., the support is removed. The removal is performed by controlled leaching-out by means of a suitable solvent or thermally, for example by thermal radiation, at elevated temperatures in the presence of oxygen. The coated support should preferably be treated within the temperature range of 490 to 600° C., such that the active composition forms a solid shell, while the support dissolves or decomposes without residue.

The invention likewise provides the oxidation of olefins to unsaturated aldehydes and corresponding acids in the presence of the inventive catalysts.

The reaction to prepare acrolein and acrylic acid is performed generally at temperatures of 250-450° C. and a pressure of 1.0-2.2 bara. The olefin, air and inert gas reactants are preferably supplied to the catalyst bed in a ratio of 1:6-9:3-18 at a loading of 2-10 mol of olefin/dm³ of catalyst bed/h.

Instead of the inert gas, it is possible to use the offgas from the reaction, from which the condensable constituents have been removed. Particularly good results are achieved when tube bundle reactors or fluidized bed reactors are used.

The inventive catalysts lead, even in the case of high specific loading, to an improved activity when used in the oxidation processes mentioned.

The invention will be illustrated hereinafter with reference to working examples. Definitions used are:

the yield (%) of the product as

(mol/h of product formed)/(mol/h of reactant supplied)* 100

the conversion of the olefin (%) as

[1−(mol/h of olefin leaving the reaction tube)/(mol/h of olefin entering the reaction tube)]*100

the selectivity (%) as

(yield of the product/conversion)*100

The invention detailed is, in order to improve understanding, described by the examples which follow, but is not restricted to these examples.

### EXAMPLES

### Example 1

A solution I was prepared by dissolving the nitrates from iron, cobalt, nickel, manganese, potassium in the proportions by mass of 23.2:47.26:29.28:0.0646:0.2067 in 3.5 litres of water and heating them to 40° C. with stirring, and adding a nitric acid solution of 0.1 mol of Sm³⁺ and 2 mol of HNO₃.

For a solution II, a solution of 2118.6 g of ammonium heptamolybdate in 2.7 l of water was prepared at 40° C.; to this end, 4.4 g of phosphoric acid and 0.42 g of Aerosil 200 (Degussa), 14 g of aluminium oxide were added to 1 l of water.

Solution II was added slowly and with intensive stirring to solution I. In a separate vessel, a further solution III consisting of 790 g of bismuth nitrate and 0.72 mol of HNO₃ was made up. Addition of this solution to the other active components afforded the coprecipitate for the preparation of the active catalyst phase.

The coprecipitate was stirred intensively for 12 hours. The resulting suspension was dried in a spray-dryer with a rotating disc at a gas inlet temperature of 350° C. The air flow was adjusted so as to obtain an exit temperature of 110+/−10° C.

This powder was treated in a forced-air oven at a temperature of 445° C. for 1 hour until a mixed oxide formed.

The mixed oxide was sprayed as an aqueous suspension through a two-substance nozzle onto a spherical Styropor support and dried at 60° C. in an air stream. To homogenize the pellets, they were circulated with rollers. To solidify the active composition applied, the resulting material was heated in the presence of oxygen at 520° C. for 5 hours.

### Example 2

The catalyst of Example 1 was contacted with a mixture of composition of 7.3% by volume of propene (chemical grade), 60% by volume of air and inert gas. At a bath temperature of 318° C. and a contact time of 2.0 s, acrolein and acrylic acid were obtained with a selectivity of 95% at a conversion of 92%.

### Comparative Example 3

The catalyst was prepared according to Example 1. The support utilized, instead of the Styropor sphere, was an alumina support which could not be removed. The resulting catalyst had the same geometric shape. At a bath temperature higher by 12° C., the reaction time had to be prolonged by the factor of 1.35 to obtain comparable conversions. The selectivity of acrolein and acrylic acid was 94%.

1. Mixed oxide catalysts which consist of hollow shapes and are of the general formula
(Mo₁₂BiₐC_{b}(Co+Ni)_{c}D_{d}EₑF_{f}G_{g}Hₕ)Oₓ (I)
in which
C: iron,
D: at least one of the elements selected from the group consisting of W, P,
E: at least one of the elements selected from the group consisting of Li, K, Na, Rb, Cs, Mg, Ca, Ba, Sr,
F: at least one of the elements selected from the group consisting of Ce, Mn, Cr, V,
G: at least one of the elements selected from the group consisting of Nb, Se, Te, Sm, Gd, La, Y, Pd, Pt, Ru, Ag, Au,
H: at least one of the elements selected from the group consisting of Si, Al, Ti, Zr,
and
a=0.5-5.0
b=0.5-5.0
c=2-15
d=0.01-5.0
e=0.001-2
f=0.001-5
g=0-1.5
h=0-800,
and
x=number which is determined by the valency and frequency of the elements other than oxygen.

2. Mixed oxide catalysts according to claim 1, comprising a gas-impermeable hollow shape.

3. Mixed oxide catalysts according to claim 1, comprising a gas-permeable hollow shape.

4. Mixed oxide catalysts according to claim 1, comprising a hollow shape consisting of a plurality of layers.

5. Mixed oxide catalysts according to claim 1, comprising hollow spheres having a diameter of 0.5-5 mm.

6. Process for preparing mixed oxide catalysts consisting of hollow shapes according to claim 1, comprising mixing solutions of compounds of the elements present in the mixed oxide catalysts of the formula I, preparing coprecipitates, isolating the resulting solid, optionally drying and calcining it, and applying the resulting finely divided solid, optionally together with a binder, in the form of a suspension as a layer to a support which consists of organic material, and removing this organic material during or after the solidification of the layer applied to it.

7. Process according to claim 6, in which the support has the shape of a sphere.

8. Process according to claim 6, in which the suspension is prepared using a calcined or partly calcined fine solid whose mean particle size distribution is between 0.01 and 80 μM.

9. Process according to claim 6, in which the mean particle size distribution of the spray-dried powder, if appropriate through grinding, is between 0.1 and 50 μm.

10. Process according to claim 6, in which the mean particle size distribution of the calcined or partly calcined powder, if appropriate through grinding, is adjusted to a distribution of 0.01 to 30 μm.

11. Process according to claim 6, in which the catalyst precursor, after the application of the finely divided solid, is dried, which is followed by a thermal treatment in the temperature range of 450 to 600° C. in the presence of oxygen.

12. Process for preparing aldehydes and acids by oxidizing olefins or methylated aromatics with air or oxygen in the presence of inert gases, steam or offgases from the reaction at elevated temperatures, characterized in that a catalyst which consists of hollow shapes and is of the general formula
(Mo₁₂BiₐC_{b}(Co+Ni)_{c}D_{d}EₑF_{f}G_{G}Hₕ)Oₓ (I)
is used, in which
C: iron,
D: at least one of the elements selected from the group consisting of W, P,
E: at least one of the elements selected from the group consisting of Li, K, Na, Rb, Cs, Mg, Ca, Ba, Sr,
F: at least one of the elements selected from the group consisting of Ce, Mn, Cr, V,
G: at least one of the elements selected from the group consisting of Nb, Se, Te, Sm, Gd, La, Y, Pd, Pt, R, Ag, Au,
H: at least one of the elements selected from the group consisting of Si, Al, Ti, Zr,
and
a=0-5.0
b=0.5-5.0
c=2-15
d=0.01-5.0
e=0.001-2
f=0.001-5
g=0-1.5
h=0-800,
and
x=number which is determined by the valency and frequency of the elements other than oxygen.

13. Process for preparing aldehydes and acids by oxidizing olefins or methylated aromatics with air or oxygen in the presence of inert gases, steam or offgases from the reaction at elevated temperatures, in which catalysts according to claim 1 is used.

14. Process according to claim 12, characterized in that acrolein and acrylic acid are obtained from propene.

15. Process according to claim 12, characterized in that methacrolein and methacrylic acid are obtained from isobutene.

16. Process according to claim 12, characterized in that benzaldehyde and benzoic acid are obtained from toluene.

17. Process according to claim 12, characterized in that the catalyst is contacted with a reaction gas mixture which comprises olefin or methylated aromatics, air, inert gases in a ratio of 1:6-9:3-18.
